(19) United States
(12) Patent Application Publication
(10) Pub. No.: US 2025/0057790 A1
(43) Pub. Date: Feb. 20, 2025

(54) AN INJECTABLE PHARMACEUTICAL COMPOSITION FOR THE TREATMENT OF RESPIRATORY DISEASES IN ANIMALS

(71) Applicant: Intervet Inc., Rahway, NJ (US)

(72) Inventors: Chen-Chao Wang, West Windsor, NJ (US); Thorsten Meyer, Wiesbaden (DE); Ralf Warrass, Alzey (DE); Joachim Ullrich, Stadecken-Elsheim (DE)

(73) Assignee: Intervet Inc., Rahway, NJ (US)

(21) Appl. No.: 18/721,947

(22) PCT Filed: Dec. 23, 2022
(86) PCT No.: PCT/EP2022/087700

§ 371 Date: Jun. 20, 2024

(30) Foreign Application Priority Data
Dec. 24, 2021 (EP) 21217704.2

(51) Int. Cl. A61K 31/166; A61K 9/00; A61K 47/10; A61K 47/12; A61K 47/14; A61P 31/04

(52) U.S. Cl. CPC A61K 31/166; A61K 9/0019; A61K 47/10; A61K 47/12; A61K 47/14; A61P 31/04

(57) ABSTRACT

An injectable pharmaceutical composition for treating a bacterial infection in an animal comprising an effective amount of a compound of Formula (I) or salt thereof, and a pharmaceutically acceptable carrier, wherein the injectable pharmaceutical composition is both efficacious and safe.

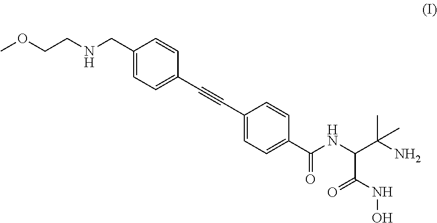 [SMILES: COCCNCc1ccc(C#Cc2ccc(C(=O)NC(C(=O)NO)C(C)(C)N)cc2)cc1]

### BACKGROUND

WO2018/115432 discloses compounds for use in the treatment of respiratory diseases of animals, especially Bovine or Swine Respiratory disease.

Bovine respiratory disease (BRD) is the most common and costly disease affecting beef cattle in the world. Bovine respiratory disease (BRD) has a multifactorial etiology and develops as a result of complex interactions between environmental factors, host factors, and pathogens. Environmental factors (e.g., weaning, transport, commingling, crowding, inclement weather, dust, and inadequate ventilation) serve as stressors that adversely affect the immune and nonimmune defense mechanisms of the host. In addition, certain environmental factors (e.g., crowding and inadequate ventilation) can enhance the transmission of infectious agents among animals. It is a complex, bacterial infection that causes Enzootic pneumonia in calves and other bovine animals and can possibly be fatal. The infection is usually a sum of three codependent factors: Stress, an underlying viral infection, and a new bacterial infection. The diagnosis of the disease is complex since there are multiple possible causes.

The term swine respiratory disease (SRD) was used to describe pneumonia of multiple etiology causing clinical disease and failure to gain weight later in the finishing process (15 to 20 weeks of age).

Injection site lesions can occur after administering animal health products. Injection site lesions are often scar tissue that forms in the muscle or subcutaneous tissue following an injection. Packers will trim injection site lesions from the carcass, often trimming muscle tissue. Injection site lesions pose beef quality issues and must be prevented. See Imer, et al., “Cull Cow Beef Quality Issues: Injection Sties and Abscesses” AN308, Department of Animal Sciences, UF/IFAS Extension, original publication date October 2014, reviewed October 2017, https://edis.ifas.ufl.edu.

An injectable pharmaceutical composition that is an effective treatment of respiratory diseases in animals and which does not produce unacceptable injection site damage when administered is needed.

### SUMMARY OF THE INVENTION

The invention concerns an injectable pharmaceutical composition comprising an effective amount of a compound of Formula (I)

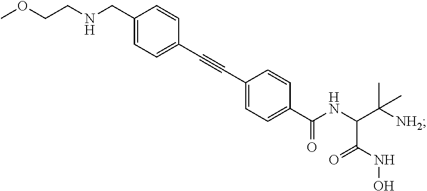 [SMILES: COCCNCc1ccc(C#Cc2ccc(C(=O)NC(C(=O)NO)C(C)(C)N)cc2)cc1]

or pharmaceutically acceptable salt thereof,
and a pharmaceutically acceptable carrier.

### DESCRIPTION OF THE FIGURES

FIG. 1 displays the injection site reaction volume data of Compounds A and B.

FIG. 2 displays the injection site reaction volume data for Compounds A and C.

### DETAILED DESCRIPTION

An injectable pharmaceutical composition that is an effective treatment of respiratory diseases such as Bovine Respiratory disease (BRD) and Swine Respiratory disease (SRD) and which does not produce unacceptable injection site damage when administered has been developed.

It has been determined that the compounds of Formula (I) are effective against the bacteria that are known to cause respiratory diseases in animals. Furthermore, the compounds of Formula (I) can be delivered to the blood and lungs of animals in acceptable concentrations when administered in the injectable pharmaceutical composition of the claimed invention. Moreover, when the compounds of Formula (I) are administered to animals in an injectable pharmaceutical composition, there is minimal injection site irritation and any irritation that does occur resolves or decreases to an acceptable level within a few days after administration.

Compound (A) is an enantiomer of the compounds of Formula (I)

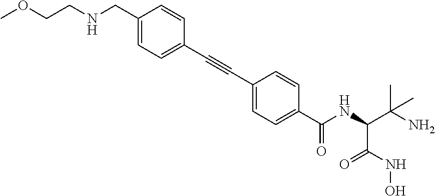 [SMILES: COCCNCc1ccc(C#Cc2ccc(C(=O)N[C@H](C(=O)NO)C(C)(C)N)cc2)cc1]

Compound (A) was found to have activity against the microbes that cause respiratory disease in animals and to be bioavailable in effective amounts in the blood and lungs of animals. When administered to animals by injection, compound (A) was tolerated at the injection site. This is not the case with other compounds with structures like Compound (A). While Compounds (B) and (C) were also active against the microbes that cause respiratory disease in animals, it has been determined that injectable pharmaceutical compositions containing Compounds (B) and (C) when administered to cattle cause unacceptable injection site reactions. (See Example 4 below).

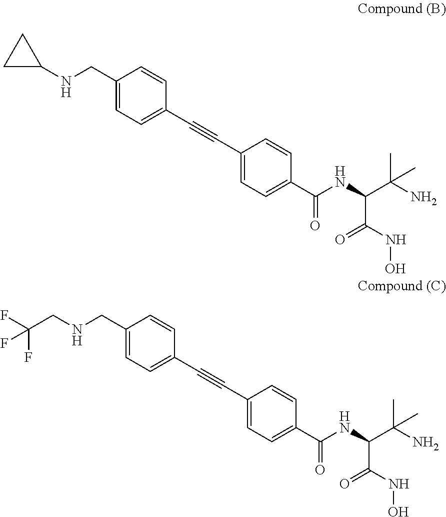 [SMILES: CC(C)(N)[C@H](NC(=O)c1ccc(C#Cc2ccc(CNC3CC3)cc2)cc1)C(=O)NO ; CC(C)(N)[C@H](NC(=O)c1ccc(C#Cc2ccc(CNCC(F)(F)F)cc2)cc1)C(=O)NO]

These results were unexpected.

Pharmaceutically acceptable salt means an acid or base salt of the compounds of the invention that is of sufficient purity and quality for use in the formulation of a composition or medicament of the present invention and are tolerated and sufficiently non-toxic to be used in a pharmaceutical preparation. Suitable pharmaceutically acceptable salts include acid addition salts which may, for example, be formed by reacting the drug compound with a suitable acid such as hydrobromic acid, hydrochloric acid, sulfuric acid, maleic acid, succinic acid, acetic acid, benzoic acid, citric acid, tartaric acid, carbonic acid or phosphoric acid.

Pharmaceutically acceptable carriers are materials that can transport the active ingredient(s) to the location where they are needed on or in the patient and are compatible with the other components of the formulation.

Pharmaceutically acceptable carriers may form solutions or suspensions with the active ingredient(s). Salts of active ingredients are often suspended in oily carries. Free base forms of the active ingredient are often dissolved in aqueous carriers.

In injectable pharmaceutical compositions that are suspensions, the pharmaceutically acceptable carriers can be an oil. In some embodiments, the oil can be a fatty acid ester. Fatty acid esters (FAEs) are a type of ester that result from the combination of a fatty acid with an alcohol. When the alcohol component is glycerol, the fatty acid esters produced can be monoglycerides, diglycerides, or triglycerides. Preferably the fatty acid ester is a medium chain triglyceride (MCT).

Medium chain triglycerides are oily pharmaceutically acceptable carriers can be any triglyceride with a 2-3 fatty acids with an aliphatic tail of between 6-12 carbon atoms. More specifically, MCTs comprise of either triglycerides of the C₈-C₁₀ fatty acids, or propylene glycoldiesters of these fatty acids or a mixture of both triglycerides and propylene glycol diesters. Preferably MCTs consist of a mixture of triglycerides of saturated fatty acids, mainly of caprylic acid (C₈H₁₆O₂) and capric acid (C₁₀H₂₀O₂). USP-NF, Interim Revision Announcement Official Mar. 1, 2019. These are conveniently prepared by the commercial fractionating of naturally occurring vegetable (e.g. coconut) oil to give mainly C8-10 fatty acids followed by esterification of these acids with a chosen alcohol. Fractionated vegetable oil having the desired composition is commercially available. Proprietary examples of such MCT oils are Miglyol® 812 as capric/caprylic triglycerides and Miglyol® 840 as propylene glycol dicaprylate/caprate.

The concentration of active ingredient in the injectable compositions is from about 1 to about 70% (by w/v). In some such embodiments, is from about 1 to about 50% (w/v), or from about 10 to about 50% (w/v). In other embodiments, the concentration is from about 35 to about 65% (w/v), from about 40 to about 60% (w/v), from about 45 to about 55% (w/v), or about 50% (w/v).

In the injectable pharmaceutical composition of current invention, the amount of the compound of Formula (I) is between about 10% and about 35% w/v, between about 15% and about 30% w/v, between about 10% and about 25% w/v, between about 20% and about 30% or about 25% w/v of the composition.

The injectable pharmaceutical compositions of the subject invention can deliver the active ingredient (the compounds of Formula (I)) in a dose in mg of active ingredient to kg of body weight of the animal of about 5 mg/kg to about 25 mg/kg, or about 10 mg/kg to about 20 mg/kg or about 10, mg/kg or about 15 mg/kg or about 20, mg/kg.

An injectable pharmaceutical composition according to the invention can also include a surfactant.

A surfactant is a substance when added to a liquid, reduces its surface tension, thereby increasing its spreading and wetting properties. A surfactant must be partly hydrophilic (water-soluble) and partly lipophilic (soluble in lipids, or oils). Examples are polyethylene glycol (15)-hydroxystearate, a poloxamer, D-α-Tocopheryl polyethylene glycol 1000 succinate, polysorbate 80 and lecithin. A preferred example is polyethylene glycol (15)-hydroxystearate.

Poloxamers are block copolymers composed of a central block of hydrophobic polyoxypropylene (poly(propylene oxide)) which is connected at each end by two blocks of polyoxyethylene (poly(ethylene oxide)) which are hydrophilic (see U.S. Pat. No. 3,740,421 and P. Alexandridis, T. A. Hutton, Colloids Surfaces A: Physiochem. Eng. Aspects 96 (1995) pp 1-46). Examples are poloxamer 188, poloxamer 407 and poloxamer 124.

In the injectable pharmaceutical composition according to the invention that is an aqueous solutions, the carrier can be water. A co-solvent can also be incorporated in the injectable pharmaceutical composition along with the water A co-solvent is a solvent that in conjunction with another solvent can dissolve a solute. The potential co-solvent for an aqueous solution may include propylene glycols, polyethylene glycol (PEG) 200, 300 and 400, 2-pyrrolidone, N-methylpyrrolidone, N,N-dimethylacetamide, glycofural, glycerol formal, glycerin, ethanol, dimethyl sulfoxide, and diethylene monoethylether or mixtures thereof.

The injectable pharmaceutical composition can be administered via intramuscular or subcutaneous route.

The injectable pharmaceutical composition can be administered to treat bacterial infections in animals. These compositions can be administered to treat respiratory disease in animals.

Bacterial agents that are frequently associated with respiratory diseases in cattle such as BRD are Mannheimia haemolytica, Pasteurella multocida, Histophilus somni, and Mycoplasma bovis. Actinobacillus pleuropneumoniae is a gram-negative bacterium is the most common cause of pleuropneumonia in pigs. P. multocida is a gram-negative bacterium which is a cause of atrophic rhinitis and pneumonia in pigs and pneumonia in ruminants. Bordetella bronchiseptica is a gram-negative bacterium that causes rhinitis and mild to moderate turbinate atrophy and predisposes to infection with toxigenic strains of P. multocida which causes the progressive form of atrophic rhinitis. Glaesserella parasuis is a Gram-negative bacterium that causes Glässer's disease, a common pathology found in young pigs characterized by polyarthritis, polyserositis, and meningitis.

The animals may be livestock, specifically cattle or swine.

Exemplary animals include but are not limited to members of the biological subfamily Bovinae which includes medium- to large-sized ungulates such as domestic dairy and beef cattle, bison, African buffalo, the water buffalo, etc. The animals may be so-called livestock raised in an agricultural setting for the production of dairy products or meat; or may be raised to perform work; or may be in another setting, e.g. in a zoo, animal reserve, etc., or raised for some other reason, e.g. as pets, show animals, for breeding purposes, etc.

Especially preferred is the use of the injectable pharmaceutical compositions of the current invention in beef cattle. Beef cattle are cattle raised for meat production (as distinguished from dairy cattle, used for milk production). There are three main stages in beef production: cow-calf operations, backgrounding, and feedlot operations. Especially preferred is the use of the injectable pharmaceutical compositions of the current invention in beef cattle in feedlot (feedyard) operations. The injectable pharmaceutical compositions of the invention can be used in beef (and dairy) cattle of every age, in calf, heifers, steer, or cows. The injectable pharmaceutical compositions of the invention can be used in animals of different weight, including calves of between 80 and 150 kg as well as heavy animals of a weight higher than 350 kg.

Other exemplary animals that can be treated with the compositions of the current invention are small ruminants, such a sheep or goats or pseudoruminants, such as e.g. camels or lamas.

The injectable pharmaceutical compositions of the current invention can be alternatively used to treat Swine respiratory disease (SRD), that is a disease of animals of the family Suidae, commonly called pigs or swine. The compositions of the current invention can be administered in general to all swine animals; to sucker, weaner, boars, barrows, gilts or sows. The injectable pharmaceutical compositions can be used in one or more of the phases of swine farming for meat: suckling pigs, feeder pigs, grower, and finisher pigs or in backfatter pigs. Alternatively, the injectable pharmaceutical compositions can be used in breeding stocks, i.e. in breeding sows, gilts or boars or the offspring of such animal as replacement breeding stock.

In one embodiment, the animal that is treated is a bovine animal and the disease that is treated is BRD.

In another embodiment the animal is a suidae (porcine) animal and the disease that is treated is SRD.

The injectable pharmaceutical compositions of the current invention can be used to treat diseased animals that display clinical symptoms of Bovine Respiratory disease or Swine respiratory disease.

In one embodiment the injectable pharmaceutical composition of the current invention is used to treat respiratory diseases such as enzootic pneumonia of lambs and/or adult sheep (ewes, rams) that are kept for meat or as breeding stock. Enzootic pneumonia is an acute infectious disease of sheep characterized by fever, nasal discharge, pneumonitis and pleuritis.

The injectable pharmaceutical compositions of the current invention can additionally or alternatively be used to treat animals with subclinical infections with Pasteurella spp., Mannheimia spp. and Histophilus spp. infections. A subclinical infection is nearly or completely asymptomatic (no disease signs or symptoms) and subclinical infections are mainly detected at the slaughterhouse when checking the lungs for lesions. However, subclinical BRD or SRD infection are commercially very relevant, because they result in lower average daily weight gains of infected animals that are additionally a source of infection for their contact animals.

In addition to treatment purposes, the injectable pharmaceutical compositions of the invention are also suitable for metaphylactic use. For example, in case of an outbreak of Bovine Respiratory disease or Swine respiratory disease, administration of the injectable pharmaceutical compositions of the current invention to non-affected (or subclinical infected) animals, especially those which are in close contact with those showing clinical signs of disease, could prevent the spread of the infection. The injectable pharmaceutical compositions of the invention are also useful for metaphylactic treatment of Bovine Respiratory disease in animals in feedlots,

In addition, prophylactic treatment might be undertaken in bovines considered to be vulnerable to infection and/or in whom infection could have grave consequences, e.g. calves, show cattle, pregnant females, prize bulls or boars, etc., whether or not an outbreak of the disease is known to have occurred. Another option is the prophylactic administration of the injectable pharmaceutical compositions according to the current invention in animals before shipping and other stress inducing events to prevent outbreak of the disease in such animals.

The same concept of prophylactic or metaphyclactic treatment, as described in the preceding paragraph applies to swine animals at risk for SRD.

The concentration of the compound of Formula (I) in the injectable pharmaceutical composition according to this invention is sufficient to provide the desired therapeutically effective amount in a volume that is acceptable for parenteral (subcutaneous) administration and allows an injection volume of less than 20 ml, preferably less than 10 ml per injection site.

The injectable pharmaceutical composition according to this invention are packaged into an appropriate container containing single or multiple doses ready for administration (ready to use—RTU.

### ADDITIONAL EMBODIMENTS

Embodiment 1. An injectable pharmaceutical composition comprising an effective amount of a compound of Formula (I)

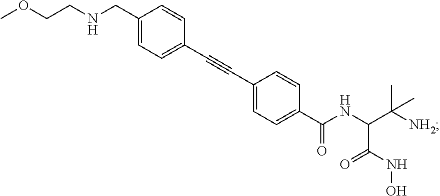 [SMILES: COCCNCc1ccc(C#Cc2ccc(C(=O)NC(C(=O)NO)C(C)(C)N)cc2)cc1]

or a pharmaceutically acceptable salt thereof,
and a pharmaceutically acceptable carrier.

Embodiment 2. The injectable pharmaceutical composition of embodiment 1, wherein the compound of Formula (I) is the compound of Formula (A)

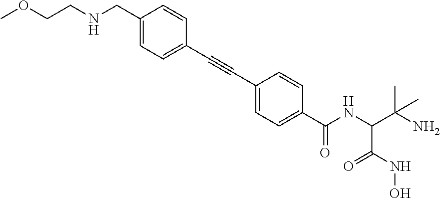 [SMILES: COCCNCc1ccc(C#Cc2ccc(C(=O)NC(C(=O)NO)C(C)(C)N)cc2)cc1]

Embodiment 3. The injectable pharmaceutical composition of embodiments 1 or 2, wherein the compound is the dihydrochloride salt of the compound of Formula (I).

Embodiment 4. The injectable pharmaceutical composition of any one of embodiments 1-3, wherein the amount of the compound of Formula (I) is between about 10% and about 35% w/v, between about 15% and about 30% w/v, between about 10% and about 25% w/v, between about 20% and about 30% or about 25% w/v of the composition.

Embodiment 5. The injectable pharmaceutical composition of any one of embodiments 1-4, wherein the pharmaceutically acceptable carrier is a medium chain triglyceride, and the compound of Formula (I) forms a suspension in the medium chain triglyceride.

Embodiment 6. The injectable pharmaceutical composition of embodiment 5, wherein the composition further comprises a surfactant.

Embodiment 7. The injectable pharmaceutical composition of embodiment 6, wherein the amount of the surfactant is between about 0.01% w/v and about 1.0% w/v.

Embodiment 8. The injectable pharmaceutical composition of any one of embodiments 6-7, wherein the surfactant is a polyethylene glycol (15)-hydroxystearate, a poloxamer, a D-α-Tocopheryl polyethylene glycol 1000 succinate, a polysorbate 80 or a lecithin.

Embodiment 9. The injectable pharmaceutical composition of embodiment 8, wherein the surfactant is a polyethylene glycol (15)-hydroxystearate.

Embodiment 10. The injectable pharmaceutical composition of any one of embodiments 1-4, wherein the pharmaceutically acceptable carrier is a water, and the compound of Formula (I) forms a solution in the water.

Embodiment 11. The injectable pharmaceutical composition of embodiment 10, wherein the composition further comprises a surfactant.

Embodiment 12. The injectable pharmaceutical composition of embodiment 11, wherein the surfactant is selected from a benzyl alcohol, a poloxamer 124, a citric acid or mixtures thereof

Embodiment 13. An injectable pharmaceutical composition comprising
a) about 10% to about 35% w/v of a dihydrochloride salt of a compound of Formula (A)

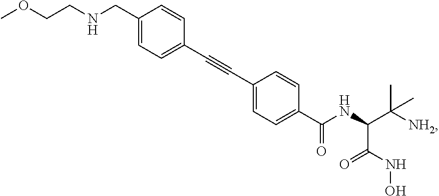 [SMILES: COCCNCc1ccc(C#Cc2ccc(C(=O)N[C@H](C(=O)NO)C(C)(C)N)cc2)cc1]

b) about 0.01 to about 1% w/v polyethylene glycol (15)-hydroxystearate and
c) a medium chain triglyceride (QS).

Embodiment 14. An injectable pharmaceutical composition comprising
a) about 10% to about 35% w/v of a compound of Formula (A)

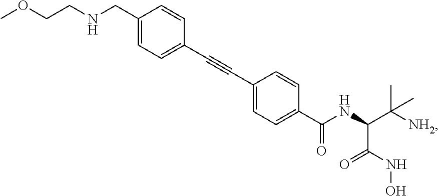 [SMILES: COCCNCc1ccc(C#Cc2ccc(C(=O)N[C@H](C(=O)NO)C(C)(C)N)cc2)cc1]

b) about 10% to about 20% w/v of a poloxamer,
c) about 4% to about 8% w/v benzyl alcohol,
d) about 5% to about 10% w/v citric acid, and
e) water (QS).

Embodiment 15. A method of treating a bacterial infection in an animal comprising administering to the animal an effective dose of the injectable pharmaceutical composition of any one of embodiments 1-14.

Embodiment 16. The method of claim 15, wherein the bacteria infection results from an infection by Mannheimia haemolytica, Pasteurella multocida, Histophilus somni, Actinobacillus pleuropneumoniae, Bordetella bronchiseptica, or Glaesserella parasuis.

Embodiment 17. The method of any one of embodiments 15-16, wherein the animal is a bovid or a swine.

Embodiment 18. A method of treating a respiratory infection in an animal comprising administering to the animal an effective dose of the injectable pharmaceutical composition of any one of embodiments 1-14.

Embodiment 19. The method of embodiment 18, wherein the respiratory infection is bovine respiratory disease, and the animal is a bovid.

Embodiment 20. The method of embodiment 18, wherein the respiratory infection is swine respiratory disease, and the animal is a swine.

Embodiment 21. An injectable pharmaceutical composition of any one of embodiments 1-14 for use in the treatment of a bacterial infection in an animal.

Embodiment 22. An injectable pharmaceutical composition for use according to embodiment 21, wherein the bacterial infection results from an infection by Mannheimia haemolytica, Pasteurella multocida, Histophilus somni, Actinobacillus pleuropneumoniae, Bordetella bronchiseptica, or Glaesserella parasuis.

Embodiment 23. An injectable pharmaceutical composition for use according to any embodiment 21 or 22, wherein the animal is a bovid or a swine.

Embodiment 24. An injectable pharmaceutical composition of any one of embodiments 1-14 for use in the treatment of a respiratory infection in an animal.

Embodiment 25. An injectable pharmaceutical composition for use according to embodiment 24, wherein the respiratory infection is bovine respiratory disease, and the animal is a bovid.

Embodiment 26. An injectable pharmaceutical composition for use according to embodiment 24, wherein the respiratory infection is swine respiratory disease, and the animal is a swine.

Embodiment 27. An injectable pharmaceutical composition for use according to any embodiment 21 to 26 comprising administering to the animal an effective dose of the injectable pharmaceutical composition.

The invention will now be further described by the following, non-limiting, examples.

### EXAMPLES

### Example 1: Injectable Pharmaceutical Composition that is a Suspension of the Dihydrochloride Salt of the Compound of Formula (A)

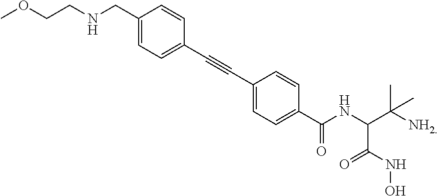 [SMILES: COCCNCc1ccc(C#Cc2ccc(C(=O)NC(C(=O)NO)C(C)(C)N)cc2)cc1]

| Ingredient | % (w/v) |
|---|---|
| Compound A | 25 |
| Polyethylene glycol (15)-hydroxystearate | 0.1 |
| (Kolliphor HS15) | |
| medium chain triglycerides (Miglyol 812) | QS to 100% |

The following procedure was used to prepare 100 mL of the above suspension injectable pharmaceutical composition:
1. Mixed 0.1 g polyethylene glycol (15)-hydroxystearate (Kolliphor HS15) into 49.9 g of medium chain triglycerides in a 150-250 mL PTFE beaker. The mixture was then warmed in a water bath to a temperature at approximately 50° C. Once the polyethylene glycol (15)-hydroxystearate dissolved, the mixture was stirred until a uniform clear solution was observed.
2. 25 g of the dihydrochloride salt of compound of Formula (A) and approximately 10 g of medium chain triglycerides were added to the solution of step 1. The resultant mixture was homogenized using an IKA homogenizer (Model T25 Digital Ultra Turrax,) with a 18G shaft until a uniform suspension was observed.
3. The suspension of step 2 was transferred to a 100 mL volumetric flask.
4. 5 mL of medium chain triglycerides was used to rinse the beaker and transferred to the volumetric flask. This rinsing procedure was repeated twice. An additional amount of medium chain triglycerides was added to bring the volume in the flask to 100 mL. The suspension was then mixed well by hand.
5. The suspension was transferred to a clean PTFE beaker and homogenized until the suspension was uniform.

### Example 2: Injectable Pharmaceutical Composition that is a Solution of the Compound of Formula (A)

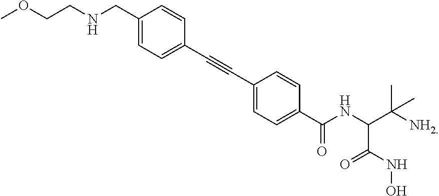 [SMILES: COCCNCc1ccc(C#Cc2ccc(C(=O)NC(C(=O)NO)C(C)(C)N)cc2)cc1]

| Ingredient | % (w/v) |
|---|---|
| Compound A | 15 |
| Citric acid anhydrate | 7 |
| Poloxamer P124 | 15 |
| Benzyl alcohol | 6 |
| Water for injection | 55 |
| 30% (w/w) citric acid solution | Quantity sufficient for pH adjustment |
| | to 4-4.5 |
| 1N Sodium hydroxide | Quantity sufficient for pH adjustment |
| solution | to 4-4.5 |
| Water for injection | QS to 100% |

The following procedure was used to prepare 100 mL of the above aqueous solution injectable pharmaceutical composition:
1. Citric acid anhydrate, poloxamer P124, benzyl alcohol and 55 g (or 55% of the target volume of finished product) water for injection were added to a volumetric flask. This mixture was stiffed using a magnetic stirrer until the solution was clear.
2. Compound A was added to the flask and mixed overnight using a magnetic stirrer. The composition was opaque or turbid. 30% citric acid solution was added drop wise with mixing until pH was in the range of 4-4.5.
3. Solution of step 2 was mixed until it was clear.
4. The solution was mixed overnight. The pH was checked and addition amounts of 30% citric acid solution and iN sodium hydroxide solution were added to adjust the pH to 4-4.5.
5. QS with water for injection. Mixed well.

### Example 3: Antimicrobial Activity of the Compounds of Formula (I)

This study determined the in vitro activity Compound (A), the enantiomer of the compound of Formula (I), against different isolates of Actinobacillus (A.) pleuropneumoniae, Bordetella (B.) bronchiseptica, Mannheimia (M.) haemolytica, Pasteurella (P.) multocida, Histophilus (H.) somni and Glaesserella parasuis collected in different European countries.

The in vitro activity was determined by measuring the minimum inhibitory concentrations (MIC) according to CLSI document VET01-A4 [1] and by calculating MIC₅₀ and MIC₉₀.

A total of 193 field isolates were tested: 31 of A. pleuropneumoniae, 20 of B. bronchiseptica, 20 of H. somni, 20 H. parasuis, 55 of M. haemolytica and 47 of P. multocida. All bacteria were isolated from the respiratory tract of cattle and swine suffering from respiratory disease. All isolates were epidemiologically unrelated as specified by the different suppliers. E. coli ATCC 25922 (ID 6105) and M. haemolytica ATCC 33396 (ID 6374) were used as reference strains for testing isolates of B. bronchiseptica, M. haemolytica and P. multocida. A. pleuropneumoniae ATCC 27090 (ID 6314) and H. somni ATCC 700025 (ID 6315) were used as reference strains for the testing isolates of A. pleuropneumoniae, H. somni and Glaesserella parasuis. Additionally, H. parasuis (G. parasuis) ATCC 19417 was included as type strain for growth control. The MICs obtained for Compound A against the reference strains are presented in Table 1.

### Culture Media and Supplements

Mueller-Hinton agar (Becton Dickinson, Lot 9224866)
Cation-adjusted Mueller-Hinton broth, (CAVHB, Becton Dickinson, Lot 8190586)
Sheep blood, defibrinated (Thermo Scientific, Lot 37091500)
Chocolate agar, GC II with IsoVitalex™ (Becton Dickinson, Lot 0147695)
GC-agar base (Becton Dickinson, Lot 6082608)
Hemoglobin solution (Becton Dickinson, Lot 7142854)
Vitox (Oxoid, Lot 2344976)
Veterinary fastidious Medium (VFM), prepared according to CLSI document VET01-A4 [1], containing:
   CAMHB (Becton Dickinson, Lot 8190586)
   Yeast extract (Sigma, Lot BCBS5470V)
   Lysed horse blood (Thermo Scientific, Lot 35875000)
   Supplement C™ (Becton Dickinson, Lot 8030960)
Deionized water

The media were prepared according to the manufacturer's instructions.

The MICs for all isolates except for H. somni and Glaesserella parasuis isolates, were determined by using the broth-microdilution method according to CLSI document VET01-A4 [1]. For isolates of H. somni and Glaesserella parasuis the agar-dilution method with chocolate agar GC II, was used. The test concentration range for all compounds was 16 μg/mL to 0.016 μg/mL.

The MIC results were interpreted according to the CLSI document VET01-S3 [2]. The MIC is the lowest concentration of antimicrobial agent that completely inhibits growth of the organisms. All wells containing test item were compared with the growth-control wells. The lowest concentration of compound at which no visible growth (i.e. no turbidity) was detected by the unaided eye was recorded as the MIC. If a “trailing endpoint” occurs, the MIC is defined as the first concentration at which a markedly reduction (about 90%) of the inoculum is observed.

For isolates tested by the agar-dilution method, results were interpreted according to the CLSI document VET01-S3 [2]. The MIC is the lowest concentration of antimicrobial agent that completely inhibits colony formation, disregarding a single colony or a faint haze caused by inoculum. The MIC₅₀ and MIC₉₀ represent the concentrations at which a minimum of 50% or 90% of the isolates tested are inhibited.

TABLE 1
Distribution of MICs in μg/mL of Compound A obtained for the isolates tested

| | | | | P. multo- | P. multo- | H. somni | | |
|---|---|---|---|---|---|---|---|---|
| | A. pleuro- | B. | M. | cida | cida | broth | H. somni | Glaesser- |
| MIC in | pneumo- | bronchi- | haemo- | (bovine | (porcine | micro- | agar | ella |
| μg/mL | niae | septica | lytica | origin) | origin) | dilution | dilution | parasuis |
| ≤0.016 | | | | 6 | 3 | | | |
| 0.032 | | | | 12 | 12 | | | |
| 0.063 | | | 2 | 6 | 6 | | | |
| 0.125 | | | 6 | 1 | 1 | 1 | | |
| 0.25 | | | 15 | | | 5 | 1 | |
| 0.5 | | | 27 | | | 8 | 1 | 2 |
| 1 | 22 | | 5 | | | 5 | 14 | 12 |
| 2 | 9 | | | | | 1 | 4 | 5 |
| 4 | | | | | | | | 1 |
| 8 | | 10 | | | | | | |
| 16 | | 10 | | | | | | |
| >16 | | | | | | | | |
| Total | 31 | 20 | 55 | 25 | 22 | 20 | 20 | 20 |
| tested | | | | | | | | |
| MIC₅₀ | 1 | 8 | 0.5 | 0.032 | 0.032 | 0.5 | 1 | 1 |
| MIC₉₀ | 2 | 16 | 0.5 | 0.063 | 0.063 | 1 | 2 | 2 |

1. Clinical and Laboratory Standards Institute (CLSI). Performance standards for antimicrobial disk and dilution susceptibility tests for bacteria isolated from animals; approved standard—fourth edition. CLSI document VET01-A4. (ISBN 1-56238-878-9). CLSI, Wayne, Pennsylvania 19087, USA, 2013.
2. Clinical and Laboratory Standards Institute (CLSI). Performance standards for antimicrobial disk and dilution susceptibility tests for bacteria isolated from animals; third informational supplement. CLSI document VET01S 3^{rd} edition. (ELECTRONIC ISBN 1-56238-908-4). CLSI, Wayne, Pennsylvania 19087, USA, 2015.

### Example 4: Injection Site Reactions

Cattle injected with injectable pharmaceutical compositions containing Compound (A) were evaluated for reactions at the injection site. These injection site reactions were compared to the injection site reactions of cattle injected with injectable pharmaceutical compositions containing Compounds (B) and (C).

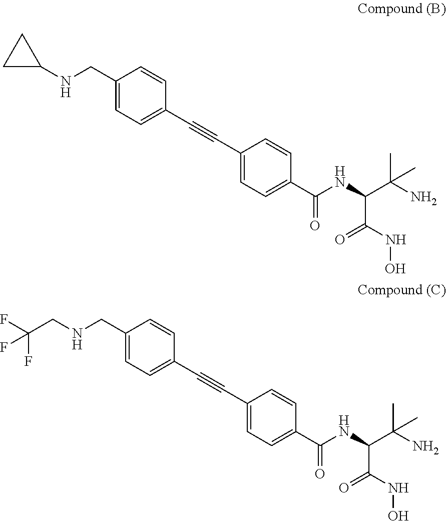 [SMILES: CC(C)(N)[C@H](NC(=O)c1ccc(C#Cc2ccc(CNC3CC3)cc2)cc1)C(=O)NO ; CC(C)(N)[C@H](NC(=O)c1ccc(C#Cc2ccc(CNCC(F)(F)F)cc2)cc1)C(=O)NO]

The compounds were formulated as aqueous solution injectable pharmaceutical compositions or as suspension injectable pharmaceutical compositions as indicated below. These formulations were prepared as described in Examples 1 and 2.

Compound (A) was formulated as both an aqueous solution using the free base form of the compound and as a suspense using the dihydrochloride salt of the compound. The composition of the aqueous solution injectable pharmaceutical composition was 15% w/v of Compound A, 15% w/v Poloxamer P124, 6% w/v Benzyl Alcohol, 7% w/v Citric Acid, and QS Water. The composition of the suspension injectable pharmaceutical composition was 15% w/v Compound A (17.5% w/v HCl salt of Compound A), 0.1% w/v Kolliphor HS 15 and QS Miglyol 812. Compound A was administered to the animals in a dose of 10 mg/kilogram of body weight.

Compound (B) was formulated as both an aqueous solution using the free base form of the compound and as a suspense using the dihydrochloride salt of the compound. The composition of the aqueous solution injectable pharmaceutical composition was 15% w/v of Compound B, 15% w/v Poloxamer P124, 6% w/v Benzyl Alcohol, 7% w/v Citric Acid, and QS Water. The composition of the suspension injectable pharmaceutical composition was 15% w/v Compound B (17.5% w/v HCl salt of Compound B), 0.1% w/v Kolliphor HS 15 and QS Miglyol 812. Compound A was administered to the animals in a dose of 10 mg/kilogram of body weight.

Compound (C) was formulated as both an aqueous solution using the free base form of the compound. The composition of the aqueous solution injectable pharmaceutical composition was 10% w/v of Compound C, 15% w/v Poloxamer P124, 6% w/v Benzyl Alcohol, 7% w/v Citric Acid, and QS Water. Compound A was administered to the animals in a dose of 10 mg/kilogram of body weight.

Injection sites were examined clinically by a trained person,

Size of the swelling is measured with a ruler to the closest 0.5 cm horizontally, vertical and depth. These three measured values were multiplied to an estimation of the volume of the square box surrounding the observed swelling.

The injection site reaction data are presented in Table 2 below

Injectable

| Pharmaceutical | | | | Average Injection site volume [cm³] | | | | |
|---|---|---|---|---|---|---|---|---|
| Composition | Dose | | D −1 | D 0 pm | D +1 | D +2 | D +3 | D +6 |
| Compound C, 10% | 10 | Mean | 0 | 344 | 1528 | 930 | 705 | 1133 |
| aqueous solution | mg/kg | | | | | | | |
| | | STD | 0 | 77 | 283 | 114 | 91 | 55 |
| Compound A, 15% | 10 | Mean | 0 | 289 | 350 | 58 | 20 | 1 |
| aqueous solution | mg/kg | | | | | | | |
| | | STD | 0 | 38 | 155 | 10 | 18 | 1 |
| Compound A, 17.5% | 10 | Mean | 0 | 66 | 293 | 104 | 18 | 5 |
| oily suspension | mg/kg | | | | | | | |
| | | STD | 0 | 58 | 136 | 56 | 30 | 8 |
| Compound B, 17.5% | 5 | Mean | 0 | 52 | 590 | 603 | 494 | 371 |
| oily suspension | mg/kg | | | | | | | |
| | | STD | 0 | 26 | 169 | 323 | 328 | 159 |
| Compound B, 17.5% | 10 | Mean | 0 | 60 | 784 | 451 | 573 | 289 |
| oily suspension | mg/kg | | | | | | | |
| | | STD | 0 | 48 | 221 | 9 | 142 | 17 |
| Compound B, 15% | 10 | Mean | 0 | 344 | 463 | 427 | 371 | 182 |
| aqueous solution | mg/kg | | | | | | | |
| | | STD | 0 | 289 | 150 | 159 | 156 | 105 |

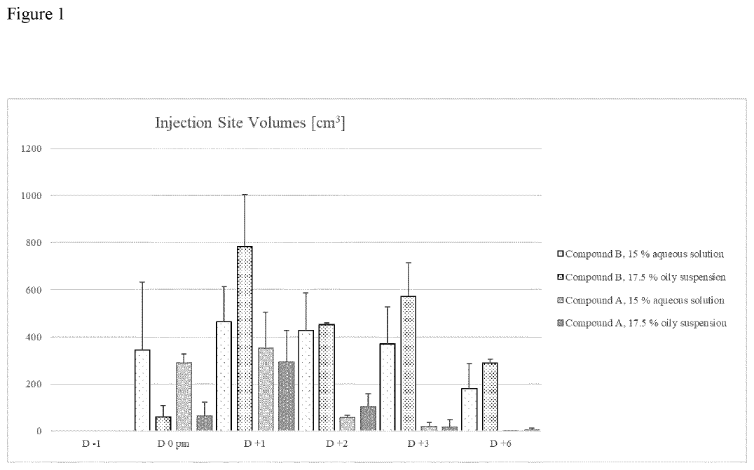

FIG. 1 displays the injection site reaction volume data of Compounds A and B.

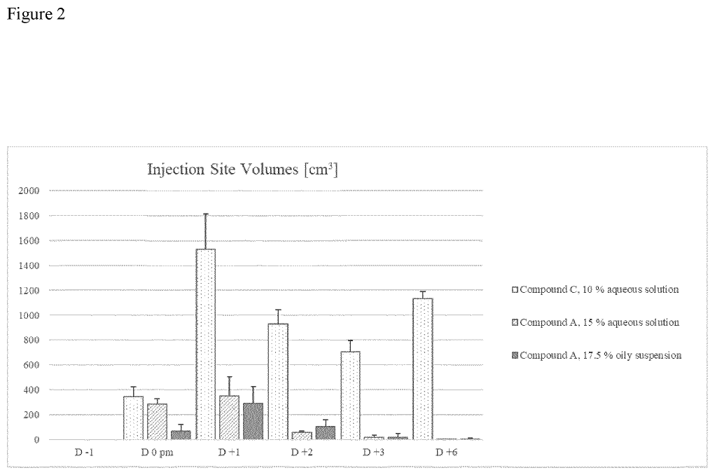

FIG. 2 displays the injection site reaction volume data for Compounds A and C.

As these data demonstrate, Compound A created minor injection reactions which were resolved by Day 6. Compounds B and C created more injection reactions than Compound A and these injection site reactions were not resolved during the duration of the study.

### Example 5—Pharmacokinetic Analysis of Compound A

This study determined the bioavailability, the pharmacokinetic profile of Compound A formulations for subcutaneous (SC) injection in the blood plasma of cattle after a single subcutaneous (SC) administration of 10 mg/kg body weight. Concentrations in the blood plasma and bronchial epithelial lining fluid concentrations were determined. Additional tissues were collected post mortem for compound concentration determination.

Compound A was formulated as described in Examples 1 and 2 above. The aqueous solution formulation was Compound A 15% w/v, 15 w/v Kollisolv P124, 6% w/v benzyl alcohol, 7% w/v citric acid, and QS Water, pH 5.2. The oily suspension formulation was Compound A 17.5% w/v, suspended with 0.1% Kolliphor HS, and QS Miglyol812.

Individual blood samples of approximately 4 mL per sample were collected from all animals at D −1 and at the following time points: 15 min, 30 min, 1 h, 2 h, 4 h, 7 h, 10 h, 24 h, 32 h, 48 h, 72 h and 142 h (D 6) after administrations. Samples were collected into K-EDTA coated Monovettes®. The samples were collected from the Vena jugularis externa. Preferred was the left side of the body, on the opposite side of administration.

The tubes were gently shaken to ensure proper mixing of blood with the anti-coagulant and placed in ice water until centrifugation. K-EDTA-Blood samples were centrifuged at approximately 4° C. and a relative centrifugal force of 2000 for 10 min. Within 2 hours after collection, two aliquots (1, 2) of approximately 0.5 mL blood plasma were pipetted off each sample into labeled plastic (polypropylene) freezer vials.

Pharmacokinetic (PK) parameters (Table 10-2) were calculated using the Phoenix WinNonlin 8.1 software. PK parameters (at least: Cmax, Tmax, AUClast, HL_Lambda_Z, VZ_F, MRTlast, CL_F and F %) to describe the PK profile after SC compound administration.

TABLE 3
Concentrations of Compound A (ng/mL) in the blood plasma of cattle
after SC administration of 10 mg/kg 15% aqueous solution

| Time after | |
|---|---|
| administration (h) | Mean |
| 0 | Not applicable |
| 0.25 | 1443 |
| 0.5 | 1627 |
| 1 | 1258 |
| 2 | 567 |
| 4 | 363 |
| 7 | 395 |
| 10 | 320 |
| 24 | 47.0 |
| 32 | 22.0 |
| 48 | 11.5 |
| 72 | 6.55 |
| 142 | Not applicable |

TABLE 4
Concentrations of Compound A (ng/mL) in the blood plasma of cattle
after SC administration of 10 mg/kg 17.5% oily suspension

| Time after | |
|---|---|
| administration (h) | Mean |
| 0 | Not applicable |
| 0.25 | 1215 |
| 0.5 | 1093 |
| 1 | 571 |
| 2 | 413 |
| 4 | 356 |
| 7 | 433 |
| 10 | 319 |
| 24 | 56.5 |
| 32 | 30.8 |
| 48 | 13.2 |
| 72 | 7.28 |
| 142 | Not applicable |

TABLE 6
Mean PK parameters for Compound A in the aqueous
solution and Compound A in the oily suspension

| | Cmax | Tmax | AUClast | T½_elim |
|---|---|---|---|---|
| Group | (ng/ml) | (h) | (h*ng/ml) | (h) |
| Comp A | 1,674.3 | 0.6 | 7,982.4 | 24.1 |
| aq. sol. | | | | |
| Comp A | 1,215.3 | 0.3 | 7,407.2 | 17.3 |
| oily susp | | | | |

In both the aqueous solution group and the oily suspension group, Compound A was detected in bovine plasma for on average 72 h. and exhibited overall a comparable PK plasma profile. Both groups showed high initial plasma levels, pronounced constant plasma plateau levels from 4-10 h after treatment, high systemic exposure in cattle and relatively short compound persistence of around 11 h in cattle.

### Example 6—Lung Tissue Study

The same animals and compositions of Compound A that were used in Example 5 were also used for the Lung tissue study. The lung was freed from the trachea and appending tissues. One half of the lung was homogenized using a food processor (Robot Coupe). Two aliquots of approximately 1 g were placed into 15 mL Falcon tubes. From the liver, kidney and a skeletal muscle (long back muscle) scissor snips from at least two locations were collected to a total mass of ca. 1 g each and placed into 15 mL Falcon tubes. Tissue samples were diluted with blank EDTA cattle plasma 1+3 (w/v) and samples were homogenized using a FastPrep™ System. After centrifugation aliquots of the supernatants were used to determine compound concentrations by a HPLC-MS/MS procedure.

TABLE 5
Concentrations in lung tissue of cattle for Compound A in
the aqueous solution and Compound A in the oily suspension

| | Mean Concentration of Compound |
|---|---|
| Group | A in Cattle Lung Tissue (ng/g) |
| Compound A in the aqueous solution | 289 |
| Compound A in the oily suspension | 285 |

Compound A was detected in
in substantial amounts in tissue samples from lungs after slaughter.

### Example 7—Efficacy Study

Efficacy of compound (A) as one-shot treatment at 20 mg/kg is shown in a naturally occurring BRD outbreak in comparison to a non-treated control group (saline) and a standard-of-care treatment (Draxxin™).

Animals are observed for 14 days post treatment. Respiratory and depression scores as well as morbidity and mortality rates are recorded.

1. An injectable pharmaceutical composition comprising an effective amount of a compound of Formula (I) 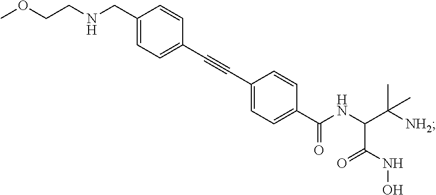 [SMILES: COCCNCc1ccc(C#Cc2ccc(C(=O)NC(C(=O)NO)C(C)(C)N)cc2)cc1]
or a pharmaceutically acceptable salt thereof,
and a pharmaceutically acceptable carrier.

2. The injectable pharmaceutical composition of claim 1, wherein the compound of Formula (I) is the compound of Formula (A).

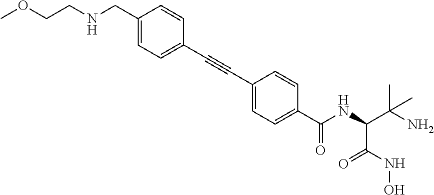 [SMILES: COCCNCc1ccc(C#Cc2ccc(C(=O)N[C@H](C(=O)NO)C(C)(C)N)cc2)cc1]

3. The injectable pharmaceutical composition of claim 1, wherein the compound is the dihydrochloride salt of the compound of Formula (I).

4. The injectable pharmaceutical composition of claim 1, wherein the amount of the compound of Formula (I) is between about 10% and about 35% w/v, between about 15% and about 30% w/v, between about 10% and about 25% w/v, between about 20% and about 30% or about 25% w/v of the composition.

5. The injectable pharmaceutical composition of claim 1, wherein the pharmaceutically acceptable carrier is a medium chain triglyceride, and the compound of Formula (I) forms a suspension in the medium chain triglyceride.

6. The injectable pharmaceutical composition of claim 5, wherein the composition further comprises a surfactant.

7. The injectable pharmaceutical composition of claim 6, wherein the amount of the surfactant is between about 0.01% w/v and about 1.0% w/v.

8. The injectable pharmaceutical composition of claim 6, wherein the surfactant is a polyethylene glycol (15)-hydroxystearate, a poloxamer, a D-α-Tocopheryl polyethylene glycol 1000 succinate, a polysorbate 80 or a lecithin.

9. The injectable pharmaceutical composition of claim 8, wherein the surfactant is a polyethylene glycol (15)-hydroxystearate.

10. The injectable pharmaceutical composition of claim 1, wherein the pharmaceutically acceptable carrier is a water, and the compound of Formula (I) forms a solution in the water.

11. The injectable pharmaceutical composition of claim 10, wherein the composition further comprises a surfactant.

12. The injectable pharmaceutical composition of claim 11, wherein the surfactant is selected from a benzyl alcohol, a poloxamer 124, a citric acid or mixtures thereof

13. An injectable pharmaceutical composition comprising
a) about 10% to about 35% w/v of a dihydrochloride salt of a compound of Formula (A) 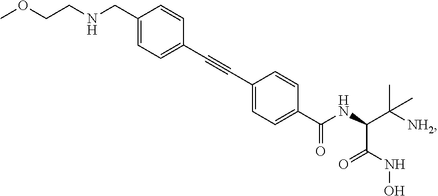 [SMILES: COCCNCc1ccc(C#Cc2ccc(C(=O)N[C@H](C(=O)NO)C(C)(C)N)cc2)cc1]
b) about 0.01 to about 1% w/v polyethylene glycol (15)-hydroxystearate and
c) a medium chain triglyceride (QS).

14. An injectable pharmaceutical composition comprising
a) about 10% to about 35% w/v of a compound of Formula (A) 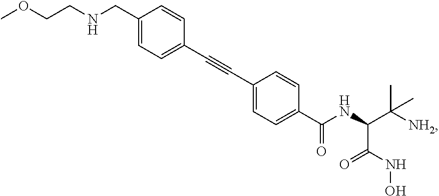 [SMILES: COCCNCc1ccc(C#Cc2ccc(C(=O)N[C@H](C(=O)NO)C(C)(C)N)cc2)cc1]
b) about 10% to about 20% w/v of a poloxamer,
c) about 4% to about 8% w/v benzyl alcohol,
d) about 5% to about 10% w/v citric acid, and
e) water (QS).

15. A method of treating a bacterial infection in an animal comprising administering to the animal the injectable pharmaceutical composition of claim 1.
